(19) United States
(12) Patent Application Publication
(10) Pub. No.: US 2025/0333522 A1
(43) Pub. Date: Oct. 30, 2025

(54) COMPOSITIONS AND METHODS FOR TREATING PAIN IN SUBJECTS WITH RHEUMATOID ARTHRITIS

(71) Applicants: SANOFI BIOTECHNOLOGY, Gentilly (FR); REGENERON PHARMACEUTICALS, INC., Tarrytown, NY (US)

(72) Inventors: Susan Boklage, Tarrytown, NY (US); Toshio Kimura, Tarrytown, NY (US); Stefano Fiore, Guttenberg, NJ (US); Gregory St John, Tarrytown, NY (US); Wenhui Wei, Tarrytown, NY (US); Vivian Bykerk, New York, NY (US)

(21) Appl. No.: 19/175,226

(22) Filed: Apr. 10, 2025

Related U.S. Application Data
(63) Continuation of application No. 16/891,984, filed on Jun. 3, 2020, now Pat. No. 12,297,279.
(60) Provisional application No. 62/857,247, filed on Jun. 4, 2019.
(60) Provisional application No. 62/930,966, filed on Nov. 5, 2019.

(30) Foreign Application Priority Data
Feb. 27, 2020 (EP) 20305191.7

(51) Int. Cl. C07K 16/28; A61K 9/00; A61P 29/00; C07K 16/24

(52) U.S. Cl. CPC C07K 16/2866; A61K 9/0019; A61P 29/00; C07K 16/241; C07K 2317/565

(57) ABSTRACT

The present disclosure relates to the use of an anti-IL6 receptor antibody for treating unacceptable pain in subjects with rheumatoid arthritis. Subjects with unacceptable pain can have, e.g., refractory pain or strict refractory pain.

### CROSS-REFERENCE TO RELATED APPLICATIONS

This application is a continuation of U.S. patent application Ser. No. 16/891,984, filed Jun. 3, 2020, which claims the benefit of U.S. Provisional Patent Application Ser. Nos. 62/857,247, filed Jun. 4, 2019, 62/930,966, filed Nov. 5, 2019, and European Patent Application No. 20305191.7, filed Feb. 27, 2020, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Apr. 10, 2025, is named 764069_SA9-269CON_ST26.xml and is 14,810 bytes in size.

### FIELD

The present disclosure relates to the field of therapeutic treatment of unacceptable pain in subjects who have or who have had rheumatoid arthritis.

### BACKGROUND

Pain is a core-set domain and a troubling symptom to patients with rheumatoid arthritis (RA), and may be directly related to inflammation. Unacceptable pain (UP) levels may persist in patients despite receiving treatment-induced inflammation control (IC), i.e. refractory pain (RP).

Sarilumab is an interleukin-6 receptor antagonist for treatment of adults with moderately to severely active RA with an inadequate response or intolerance to one or more disease-modifying antirheumatic drugs (DMARDs).

### SUMMARY

This disclosure provides methods and compositions for treating unacceptable pain in a subject who has rheumatoid arthritis. In various embodiments, treating the subject comprises administering a therapeutically effective amount of an antibody that specifically binds IL-6R.

In various embodiments, the antibody that specifically binds to the IL-6 receptor comprises a heavy chain variable region sequence of SEQ ID NO: 1 and a light chain variable region sequence of SEQ ID NO: 2

In various embodiments, the antibody comprises a heavy chain variable region (VH) and a light chain variable region (VL), wherein the VH comprises the three complementarity determining regions (CDRs) found within the sequence of SEQ ID NO:1 and wherein the VL comprises the three CDRs found within the sequence of SEQ ID NO:2. In various embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8.

In various embodiment, the antibody is sarilumab.

In various embodiments, the subject has refractory pain. In various embodiments, the subject has UP despite treatment-induced inflammation control. In various embodiments, the subject has UP despite a reduction in inflammation of at least about 10%, 15%, 20%, 25%, 30%, 35%, 40%, 45%, or 50% compared to when the subject was first treated with a DMARD. In various embodiments, the subject has UP despite a reduction in inflammation of from 10% to 25%, from 25% to 50%, from 50% to 75%, from 75% to 95%, or from 75% to 100% compared to when the subject was first treated with a DMARD. In various embodiments, the subject has UP despite a reduction in inflammation of at least about 10%, 15%, 20%, 25%, 30%, 35%, 40%, 45%, or 50% compared to when the subject was first treated with one or more DMARDs other than sarilumab. In various embodiments, the subject has UP despite a reduction in inflammation of from 10% to 25%, from 25% to 50%, from 50% to 75%, from 75% to 95%, or from 75% to 100% compared to when the subject was first treated with one or more DMARDs other than sarilumab. In various embodiments, the subject has strict refractory pain. In various embodiments, the subject experiences a reduction in visual analog scale (VAS) of less than 40 after 24 or 52 weeks of treatment.

In various embodiments, the antibody is administered subcutaneously. In various embodiments, the antibody is administered every week, or every two weeks. In various embodiments, the antibody is administered once every week or once every two weeks.

In various embodiments, the antibody is administered at a dose from about 150 mg to 200 mg. In various embodiments, the antibody is administered at a dose of about 150 mg or about 200 mg. In various embodiments, the antibody is administered at a dose from 150 mg to 200 mg. In various embodiments, the antibody is administered at a dose of 150 mg or 200 mg.

In various embodiments, the subject has rheumatoid arthritis. In various embodiments, the subject has moderately to severely active rheumatoid arthritis. In various embodiments, the subject has moderately active rheumatoid arthritis. In various embodiments, the subject has severely active rheumatoid arthritis.

In various embodiments, a subject has a Disease Activity Score (DAS) of from 3.2 to 5.1. In various embodiments, a subject has a DAS of greater than 5.1. In various embodiments, the subject has a DAS of 3.2 or more. In various embodiments, the subject has a DAS of from 5 to 6, from 5 to 7, from 5 to 8, from 5 to 9, from 5 to 10, or from 7.5 to 10. The DAS for a subject can readily be calculated by those in the art. Non-limiting descriptions relating to DAS are provided in Fransen and van Riel (Clin Exp Rheumatol. 2005 September-October;23 (5 Suppl 39):S93-9), the entire content of which is incorporated herein by reference.

In various embodiments, no other disease modifying antirheumatic drug (DMARD) is administered with the antibody. In various embodiments, at least one other DMARD is administered to the subject. In various embodiments, at least one other DMARD is administered to the subject concurrently with or at the same time as the antibody. In various embodiments, the subject was previously ineffectively treated for rheumatoid arthritis by administering at least one DMARD different from the antibody. In various embodiments, the subject is intolerant of one or more DMARDs, or wherein the subject is considered an inappropriate candidate for continued treatment with one or more DMARDs.

In various embodiments, the DMARD is an sDMARD. In various embodiments, the DMARD is methotrexate. In various embodiments, the DMARD is a TNF antagonist. In various embodiments, the TNF antagonist is selected from etanercept, infliximab, adalimumab, golimumab and certolizumab pegol.

In another aspect, provided herein is a method for treating unacceptable pain (UP) in a subject in need thereof, comprising selecting a subject who has rheumatoid arthritis and UP, and administering to the subject a therapeutically effective dose of an antibody that specifically binds IL-6 receptor.

In various embodiments, the antibody that specifically binds to the IL-6 receptor comprises a heavy chain variable region sequence of SEQ ID NO: 1 and a light chain variable region sequence of SEQ ID NO: 2

In various embodiments, the antibody comprises a heavy chain variable region (VH) and a light chain variable region (VL), wherein the VH comprises the three complementarity determining regions (CDRs) found within the sequence of SEQ ID NO:1 and wherein the VL comprises the three CDRs found within the sequence of SEQ ID NO:2. In various embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8.

In various embodiment, the antibody is sarilumab.

In various embodiments, the subject has refractory pain. In various embodiments, the subject has treatment induced strict refractory pain. In various embodiments, the subject experiences a reduction in visual analog scale (VAS) of less than 40 after 24 or 52 weeks of treatment.

In various embodiments, the antibody is administered subcutaneously. In various embodiments, the antibody is administered every week, or every two weeks. In various embodiments, the antibody is administered once every week or once every two weeks.

In various embodiments, the antibody is administered at a dose from about 150 mg to 200 mg. In various embodiments, the antibody is administered at a dose of about 150 mg or about 200 mg. In various embodiments, the antibody is administered at a dose from 150 mg to 200 mg. In various embodiments, the antibody is administered at a dose of 150 mg or 200 mg.

In various embodiments, the subject has rheumatoid arthritis. In various embodiments, the subject has moderately to severely active rheumatoid arthritis. In various embodiments, the subject has moderately active rheumatoid arthritis. In various embodiments, the subject has severely active rheumatoid arthritis.

In various embodiments, no other disease modifying antirheumatic drug (DMARD) is administered with the antibody. In various embodiments, at least one other DMARD is administered to the subject. In various embodiments, at least one other DMARD is administered to the subject concurrently with or at the same time as the antibody. In various embodiments, the subject was previously ineffectively treated for rheumatoid arthritis by administering at least one DMARD different from the antibody. In various embodiments, the subject is intolerant of one or more DMARDs, or wherein the subject is considered an inappropriate candidate for continued treatment with one or more DMARDs.

In various embodiments, the DMARD is an sDMARD. In various embodiments, the DMARD is methotrexate. In various embodiments, the DMARD is a TNF antagonist. In various embodiments, the TNF antagonist is selected from etanercept, infliximab, adalimumab, golimumab and certolizumab pegol.

In a further aspect, provided herein is an antibody for use in treating unacceptable pain in a patient in need thereof who has rheumatoid arthritis, wherein the antibody specifically binds IL-6 receptor.

In various embodiments, the antibody that specifically binds to the IL-6 receptor comprises a heavy chain variable region sequence of SEQ ID NO: 1 and a light chain variable region sequence of SEQ ID NO: 2

In various embodiments, the antibody comprises a heavy chain variable region (VH) and a light chain variable region (VL), wherein the VH comprises the three complementarity determining regions (CDRs) found within the sequence of SEQ ID NO:1 and wherein the VL comprises the three CDRs found within the sequence of SEQ ID NO:2. In various embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8.

In various embodiments, the antibody is sarilumab.

In various embodiments, the subject has refractory pain. In various embodiments, the subject has treatment induced strict refractory pain. In various embodiments, the subject experiences a reduction in visual analog scale (VAS) of less than 40 after 24 or 52 weeks of treatment.

In various embodiments, the antibody is administered subcutaneously. In various embodiments, the antibody is administered every week, or every two weeks. In various embodiments, the antibody is administered once every week or once every two weeks.

In various embodiments, the antibody is administered at a dose from about 150 mg to 200 mg. In various embodiments, the antibody is administered at a dose of about 150 mg or about 200 mg. In various embodiments, the antibody is administered at a dose from 150 mg to 200 mg. In various embodiments, the antibody is administered at a dose of 150 mg or 200 mg.

In various embodiments, the subject has rheumatoid arthritis. In various embodiments, the subject has moderately to severely active rheumatoid arthritis. In various embodiments, the subject has moderately active rheumatoid arthritis. In various embodiments, the subject has severely active rheumatoid arthritis.

In various embodiments, a subject has a Disease Activity Score (DAS) of from 3.2 to 5.1. In various embodiments, a subject has a DAS of greater than 5.1. In various embodiments, the subject has a DAS of 3.2 or more. In various embodiments, the subject has a DAS of from 5 to 6, from 5 to 7, from 5 to 8, from 5 to 9, from 5 to 10, or from 7.5 to 10. The DAS for a subject can readily be calculated by those in the art. Non-limiting descriptions relating to DAS are provided in Fransen and van Riel (Clin Exp Rheumatol. 2005 September-October;23 (5 Suppl 39):S93-9), the entire content of which is incorporated herein by reference.

In various embodiments, no other disease modifying antirheumatic drug (DMARD) is administered with the antibody. In various embodiments, at least one other DMARD is administered to the subject. In various embodiments, at least one other DMARD is administered to the subject concurrently with or at the same time as the antibody. In various embodiments, the subject was previously ineffectively treated for rheumatoid arthritis by administering at least one DMARD different from the antibody. In various embodiments, the subject is intolerant of one or more DMARDs, or wherein the subject is considered an inappropriate candidate for continued treatment with one or more DMARDs.

In various embodiments, the DMARD is an sDMARD. In various embodiments, the DMARD is methotrexate. In various embodiments, the DMARD is a TNF antagonist. In various embodiments, the TNF antagonist is selected from etanercept, infliximab, adalimumab, golimumab and certolizumab pegol.

### BRIEF DESCRIPTION OF THE FIGURES

FIGS. 1A-1C are graphs showing the odds ratios of three different randomized clinical trials—MOBILITY, TARGET and MONARCH. FIG. 1A illustrates the odds ratios for unacceptable pain. FIG. 1B illustrates the odds ratios for unacceptable pain despite inflammation control (IC) or inflammation control-strict (IC-strict). FIG. 1C illustrates the odds ratios for refractory pain-strict.

FIGS. 2A-2C are graphs showing descriptive analysis of pain outcomes. FIG. 2A illustrates analysis of pain outcomes in MOBILITY (Week 24 and Week 52). FIG. 2B illustrates analysis of pain outcomes in TARGET (Week 24 and Week 52). FIG. 2C illustrates analysis of pain outcomes in MONARCH (Week 24 and Week 52).

### DETAILED DESCRIPTION

This disclosure provides pharmaceutical compositions and methods of using these compositions for the treatment of unacceptable pain (UP) levels. In some embodiments, the UP persists despite inflammation control (IC). In some embodiments, the IC is biologic treatment induced. These compositions and methods include at least one antibody that specifically binds interleukin-6 receptor (hIL-6R).

As used within the Claims, the Summary, and the Detailed Description herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

It is to be noted that the term “a” or “an” entity refers to one or more of that entity; for example, “a symptom,” is understood to represent one or more symptoms. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “pain” refers to discomfort caused by intense or damaging stimuli including illness, injury, or mental anguish. In some embodiments, pain has both physical and emotional components and it is experienced as an unpleasant sensation that can range from mild, localized discomfort to agony.

The term “unacceptable pain” refers to a level indicated by a patient acceptable symptom state (PASS), a validated measure that indicates the level of acceptable pain. PASS uses a threshold of 40 mm on a visual analog scale (VAS) that ranges from 0-100 mm, meaning that VAS>40 mm indicates unacceptable pain. See Lourdudoss et al. (Dietary intake of polyunsaturated fatty acids and pain in spite of inflammatory control among methotrexate-treated early rheumatoid arthritis patients. Arthritis Care and Research. 2018; 70(2):205-212); and Pham and Tubach (Patient acceptable symptomatic state (PASS). Joint Bone Spine 2009; 76:321-3), which are each incorporated herein by reference in their entireties. In some embodiments, pain experienced in the previous week is measured. In some embodiments, pain experienced in the previous 2, 3, 4, 5, 6, 7, 8 or more weeks is measured. In some embodiments, pain experienced in the previous month is measured. In some embodiments, pain experienced in the previous, 2, 3, 4, 5, 6, 7, 8, 9, 10, 11, 12 or more months is measured. In some embodiments, pain experienced in the previous year is measured. In some embodiments, pain experienced for the previous 2, 3, 4, 5, 6, 7, 8, 9, 10 or more years is measured.

A subject experiencing “refractory pain” has rheumatoid arthritis, unacceptable pain, and a serum level of C-reactive protein (CRP) of <10 mg/L. A subject experiencing “strict refractory pain” has rheumatoid arthritis, unacceptable pain, a swollen joint count (SJC) of less than or equal to 1, and a serum level of CRP<10 mg/L. In some embodiments, the rheumatoid arthritis is moderately-to-severe active rheumatoid arthritis.

IL-6 interacts directly with the IL-6Rα subunit and the IL-6/IL-6Rα pair forms a high affinity complex with the glycoprotein 130 (gp130) subunit. IL-6Rα also exists in a soluble form, which is involved in trans-signaling and allows IL-6 to affect cells that do not express IL-6Rα including synovial cells in the joint (Rose-John et al., J Leukoc Biol. 2006; 80 (2), 227-36). Sarilumab (SAR153191), also designated as REGN88, is a recombinant IgG1 kappa monoclonal antibody of fully human sequence directed against the alpha subunit of the IL-6 receptor complex (IL-6Rα). Sarilumab is a potent and specific inhibitor of IL-6signaling. By binding to IL-6Rα with high affinity, sarilumab blocks the binding of IL-6 and interrupts the cytokine-mediated signaling cascade. In certain embodiments, interleukin-6 is a key element in the etiology of rheumatic conditions and inhibition of its signaling is a critical part of the mechanism of action of sarilumab. In ex vivo assays, sarilumab did not demonstrate antibody-dependent cellular cytotoxicity (ADCC) or complement-dependent cytotoxicity (CDC) on relevant cell types where sarilumab binding was verified by fluorescence-activated cell sorter (FACS) analysis (Committee for Medicinal Products for Human Use, Assessment Report, Apr. 27, 2017 EMA/292840/2017, available at www_dot_ema_dot_europa_dot_eu/documents/assessment_report/kevzara_epar_public_assessment_report_en_dot_pdf.

### Antibodies

The present disclosure includes methods that comprise administering to a subject an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the patient binds specifically to the extracellular domain of hIL-6R. The term “antibody”, as used herein, refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

In certain embodiments, the antibody or antibody fragment for use in a method disclosed herein may be a monospecific antibody. In certain embodiments, the antibody or antibody fragment for use in a method disclosed herein may be a multispecific antibody, which may be specific for different epitopes of one target polypeptide or may contain antigen-binding domains specific for epitopes of more than one target polypeptide. An exemplary bi-specific antibody format that can be used in the context certain embodiments involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise an Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of certain embodiments. Any multispecific antibody format, including the exemplary bispecific antibody formats disclosed herein, may in various embodiments be adapted for use in the context of an antigen-binding fragment of an anti-IL-6R antibody using routine techniques available in the art.

The fully-human anti-IL-6R antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework residues and/or CDR residues within the VH and/or VL domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, included herein are antibodies that may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity. The term “human antibody”, as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). In certain embodiments, these forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105, incorporated by reference in its entirety) to levels typically observed using a human IgG1 hinge. The instant disclosure encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-6R, as used herein, includes antibodies that bind IL-6R (e.g., human IL-6R) or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. In some embodiments, the antibody binds IL-6R (e.g., human IL-6Rα) with a KD of from about 0.1 nM to about 1000 nM or from about 1 nM to about 100 nM. In some embodiments, the antibody binds IL-6R (e.g., human IL-6Rα) with a KD of from about 1 pM to about 100 pM or from about 40 pM to about 60 pM. Specific binding can also be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. An isolated antibody that specifically binds human IL-6R may, however, have cross-reactivity to other antigens, such as IL-6R molecules from other (non-human) species.

The term “surface plasmon resonance”, as used herein, refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

The term “KD”, as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IL-6R antibodies useful for the methods described herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-IL-6R antibodies comprising variants of any of the HCVR, LCVR, and/or CDR amino acid sequences disclosed herein having one or more conservative substitutions. For example, the present disclosure includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies described in U.S. Pat. No. 7,582,298, incorporated herein by reference in its entirety. In certain embodiments, the anti-IL-6R antibody or antigen-binding fragment thereof comprises the heavy chain complementarity determining regions (HCDRs) of a HCVR comprising the amino acid sequence of SEQ ID NO: 1 and the light chain complementarity determining regions (LCDRs) of a LCVR comprising the amino acid sequence of SEQ ID NO: 2. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-IL-6R antibody or antigen-binding fragment thereof comprises an HCVR comprising the amino acid sequence of SEQ ID NO: 1 and an LCVR comprising the amino acid sequence of SEQ ID NO: 2.

In another embodiment, the anti-IL-6R antibody or antigen-binding fragment thereof comprises a heavy chain comprising the amino acid sequence of SEQ ID NO: 9 and a light chain comprising the amino acid sequence of SEQ ID NO: 10. In some embodiments, the extracellular domain of hIL-6R comprises the amino acid sequence of SEQ ID NO: 11. According to certain exemplary embodiments, the methods of the present disclosure comprise the use of the anti-IL-6R antibody referred to and known in the art as sarilumab, or a bioequivalent thereof.

The amino acid sequence of SEQ ID NO: 1 is

EVQLVESGGGLVQPGRSLRLSCAASRFTFDDYAMHWVRQAPGKGLEWVS
GISWNSGRIGYADSVKGRFTISRDNAENSLFLQMNGLRAEDTALYYCAK
GRDSFDIWGQGTMVTVSS.

The amino acid sequence of SEQ ID NO: 2 is

DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIK.

The amino acid sequence of SEQ ID NO: 3 is RFTFDDYA.

The amino acid sequence of SEQ ID NO: 4 is ISWNSGRI.

The amino acid sequence of SEQ ID NO: 5 is AKGRDSFDI.

The amino acid sequence of SEQ ID NO: 6 is QGISSW.

The amino acid sequence of SEQ ID NO: 7 is GAS.

The amino acid sequence of SEQ ID NO: 8 is QQANSFPYT.

The amino acid sequence of SEQ ID NO: 9 is

EVQLVESGGGLVQPGRSLRLSCAASRFTFDDYAMHWVRQAPGKGLEWVS
GISWNSGRIGYADSVKGRFTISRDNAENSLFLQMNGLRAEDTALYYCAK
GRDSFDIWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK.

The amino acid sequence of SEQ ID NO: 10 is

DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

The sequence of SEQ ID NO: 11 is

MVAVGCALLAALLAAPGAALAPRRCPAQEVARGVLTSLPGDSVTLTCPG
VEPEDNATVHWVLRKPAAGSHPSRWAGMGRRLLLRSVQLHDSGNYSCYR
AGRPAGTVHLLVDVPPEEPQLSCFRKSPLSNVVCEWGPRSTPSLTTKAV
LLVRKFQNSPAEDFQEPCQYSQESQKFSCQLAVPEGDSSFYIVSMCVAS
SVGSKFSKTQTFQGCGILQPDPPANITVTAVARNPRWLSVTWQDPHSWN
SSFYRLRFELRYRAERSKTFTTWMVKDLQHHCVIHDAWSGLRHVVQLRA
QEEFGQGEWSEWSPEAMGTPWTESRSPPAENEVSTPMQALTTNKDDDNI
LFRDSANATSLPVQD.

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The disclosure in certain embodiments relates to methods comprising administering to the subject an antibody which comprises the heavy chain variable region comprising sequence SEQ ID NO: 1 and the light chain variable region comprising sequence SEQ ID NO: 2.

The disclosure provides pharmaceutical compositions comprising such antibody, and methods of using these compositions.

The antibody in various embodiments comprises the heavy chain variable region comprising sequence SEQ ID NO: 1 and the light chain variable region comprising sequence SEQ ID NO: 2 is an antibody that specifically binds human interleukin-6 receptor (hIL-6R). See international publication number WO2007/143168, incorporated herein by reference in its entirety. In one embodiment, the antibody comprises the heavy chain variable region comprising sequence SEQ ID NO: 9 and the light chain variable region comprising sequence SEQ ID NO: 10. In various embodiments, the antibody is sarilumab.

### DMARDs

Disease-modifying antirheumatic drugs (DMARDs) are drugs defined by their use in rheumatoid arthritis to slow down disease progression. DMARDs have been classified as synthetic (sDMARD) and biological (bDMARD). Synthetic DMARDs include non-exhaustively methotrexate, sulfasalazine, leflunomide, and hydroxychloroquine. Biological DMARDs include non-exhaustively adalimumab, golimumab, etanercept, abatacept, infliximab, rituximab, and tocilizumab. In some embodiments, the DMARD is a TNF antagonist. TNF antagonists include etanercept, infliximab, adalimumab, golimumab and certolizumab pegol.

### Methods of Administration and Formulations

The methods described herein comprise administering a therapeutically effective amount of an anti-IL-6R antibody to a subject. As used herein, an “effective amount” or “therapeutically effective amount” is a dose of the therapeutic that results in treatment of unacceptable pain (UP). In certain embodiments, effective amount is a dose of the therapeutic that results in treatment of UP that persists despite inflammation control (IC). As used herein, “treating” refers to causing a detectable improvement in one or more symptoms associated with UP or causing a biological effect (e.g., a decrease in the level of a particular biomarker) that is correlated with the underlying pathologic mechanism(s) giving rise to the condition or symptom(s). For example, a dose of anti-IL-6R antibody which causes a reduction in UP is deemed a “therapeutically effective amount.”

An “improvement” in a pain-associated symptom in various embodiments refers reduction in the incidence of the pain symptom which may correlate with an improvement in one or more pain-associated tests, scores or metrics (as described herein). For example, the improvement may correlate a decrease from baseline of one or more of pain criteria. In various embodiments, improvement may comprise a decrease in VAS from baseline. In various embodiments, the baseline score of VAS is ≥40 mm and is reduced to a score ≤40 mm. As used herein, the term “baseline,” with regard to a pain-associated parameter, means the numerical value of the pain-associated parameter for a patient prior to or at the time of administration of the antibody of the present invention. A detectable “improvement” can also be detected using at least one test, score or metric described herein. In various embodiments, the improvement is detected using VAS. In various embodiments, the improvement is characterized by its relation to a subject's PASS status.

In various embodiments, previous treatment with a DMARD other than an anti-IL-6R antibody (such as sarilumab) has been inadequate (e.g., as assessed by the subject and/or a physician), has been ineffective and/or has not resulted in a detectable improvement in one or more parameters or symptoms associated with pain and/or has not caused a biological effect that is correlated with the underlying pathologic mechanism(s) giving rise to the condition or symptom(s) of pain.

In various embodiments, a IL-6R antibody is administered subcutaneously. In various embodiments, the IL-6R antibody is sarilumab.

In various embodiments, a therapeutically effective amount of anti-IL-6R antibody that is administered to the subject will vary depending upon the age and the size (e.g., body weight or body surface area) of the subject as well as the route of administration and other factors well known to those of ordinary skill in the art.

In various embodiments, the dose is a fixed dose regardless of the body weight or surface area of the subject. In various embodiments, the subject is at least 18 years old. In various embodiments, the subject is from 30 to 100 years old. In various embodiments, the subject is from 35 to 100 years old. In various embodiments, the subject is from 35 to 8 years old. In various embodiments, the subject is from 40 to 70 years old.

The disclosure provides methods of using therapeutic compositions comprising anti-IL-6R antibodies or antigen-binding fragments thereof and, optionally, one or more additional therapeutic agents. The therapeutic compositions of the invention will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA, incorporated herein by reference in its entirety. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311, incorporated herein by reference in its entirety.

Various delivery systems are known and can be used to administerpharmaceutical compositions provided herein, e.g., encapsulation in liposomes, microparticles, microcapsules, receptor mediated endocytosis (see, e.g., Wu et al. (1987) J. Biol. Chem. 262:4429-4432, incorporated herein by reference in its entirety). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The IL-6R antibody can be administered subcutaneously.

The pharmaceutical composition can also be delivered in a vesicle, such as a liposome (see Langer (1990) Science 249:1527-1533, incorporated herein by reference in its entirety). In certain embodiments, the pharmaceutical composition can be delivered in a controlled release system, for example, with the use of a pump or polymeric materials. In certain embodiments, a controlled release system can be placed in proximity of the composition's target, thus requiring only a fraction of the systemic dose.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc.). As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The antibody is typically formulated as described herein and in international publication number WO2011/085158, incorporated herein by reference in its entirety.

In various embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
between about 100 mg/mL and about 200 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
at least about 130 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose, and
about 131.6 mg/mL of the antibody.

In another embodiment, the antibody is administered as an aqueous buffered solution at about pH 6.0 containing
about 21 mM histidine,
about 45 mM arginine,
about 0.2% (w/v) polysorbate 20,
about 5% (w/v) sucrose; and
about 175 mg/mL of the antibody.

In other embodiments, the antibody is administered as an aqueous buffered solution at pH 6.0 containing
21 mM histidine,
45 mM arginine,
0.2% (w/v) polysorbate 20,
5% (w/v) sucrose, and
between 100 mg/mL and 200 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
at least 130 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose, and
131.6 mg/mL of the antibody.

In another embodiment, the antibody is administered as an aqueous buffered solution at pH 6.0 containing
21 mM histidine,
45 mM arginine,
0.2% (w/v) polysorbate 20,
5% (w/v) sucrose; and
175 mg/mL of the antibody.

In various embodiments, the antibody is administered in a stable pharmaceutical formulation comprising: (i) histidine at a concentration of from 25 mM to 100 mM; (ii) arginine at a concentration of from 25 mM to 50 mM; (iii) sucrose in an amount of from 3% to 10% w/v; and (iv) polysorbate 20 in an amount of from 0.1% to 0.2%, wherein the formulation has a pH of about 5.8, about 6.0, or about 6.2, and at least 90% of the native form of the antibody is recovered after 1 month of storage at 45° C., as determined by size exclusion chromatography. In various embodiments, about 150 mg of the antibody (e.g., sarilumab) is administered to the subject.

In various embodiments, the antibody is administered in a stable pharmaceutical formulation comprising: (i) histidine at a concentration of from about 10 mM to about 25 mM; (ii) arginine at a concentration of from about 25 mM to about 50 mM; (iii) sucrose in an amount of from about 5% to about 10% w/v; and (iv) polysorbate in an amount of from about 0.1% to about 0.2% w/v, wherein the formulation has a pH of about 5.8, about 6.0, or about 6.2, and at least 90% of the native form of the antibody is recovered after 1 month of storage at 45° C., as determined by size exclusion chromatography. In various embodiments, about 150 mg of the antibody (e.g., sarilumab) is administered to the subject.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In various embodiments, the anti-IL-6R antibody (or pharmaceutical formulation comprising the antibody) can be administered to the patient using any acceptable device or mechanism. For example, the administration can be accomplished using a syringe and needle or with a reusable pen and/or autoinjector delivery device. The methods of the present disclosure include the use of numerous reusable pen and/or autoinjector delivery devices to administer an anti-IL-6R antibody (or pharmaceutical formulation comprising the antibody). Examples of such devices include, but are not limited to AUTOPEN (Owen Mumford, Inc., Woodstock, UK), DISETRONIC pen (Disetronic Medical Systems, Bergdorf, Switzerland), HUMALOG MIX 75/25 pen, HUMALOG pen, HUMALIN 70/30 pen (Eli Lilly and Co., Indianapolis, IN), NOVOPEN I, II and III (Novo Nordisk, Copenhagen, Denmark), NOVOPEN JUNIOR (Novo Nordisk, Copenhagen, Denmark), BD pen (Becton Dickinson, Franklin Lakes, NJ), OPTIPEN, OPTIPEN PRO, OPTIPEN STARLET, and OPTICLIK (Sanofi-Aventis, Frankfurt, Germany). Examples of disposable pen and/or autoinjector delivery devices having applications in subcutaneous delivery of a pharmaceutical composition of the present disclosure include, but are not limited to the SOLOSTAR pen (Sanofi-Aventis), the FLEXPEN (Novo Nordisk), and the KWIKPEN (Eli Lilly), the SURECLICK Autoinjector (Amgen, Thousand Oaks, CA), the PENLET (Haselmeier, Stuttgart, Germany), the EPIPEN (Dey, L.P.), and the HUMIRA Pen (AbbVie Inc., North Chicago, IL), to name only a few.

In various embodiments, the antibody is administered with a prefilled syringe. In various embodiments, the antibody is administered with a prefilled syringe containing a safety system. For example, the safety system prevents an accidental needle-stick injury. In various embodiments, the antibody is administered with a prefilled syringe containing an ÈRIS safety system (West Pharmaceutical Services Inc.). See also U.S. Pat. Nos. 5,215,534 and 9,248,242, incorporated herein by reference in their entireties.

In various embodiments, the antibody is administered with an auto-injector. In various embodiments, the antibody is administered with an auto-injector featuring the PUSHCLICK technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

The use of a microinfusor to deliver an anti-IL-6R antibody (or pharmaceutical formulation comprising the antibody) to a patient is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996), incorporated herein by reference in their entireties. Microinfusors are particularly useful for the delivery of large doses of therapeutic proteins contained within high concentration (e.g., about 100, 125, 150, 175, 200 mg/mL or more) and/or viscous solutions.

In various embodiments, an inadequate response to prior treatment refers to subjects whose pain is not well controlled after receiving the prior treatment at the maximum tolerated typical dose. In an embodiment, an inadequate response to prior treatment refers to subjects who have moderate or high disease activity and features of poor prognosis despite prior treatment. In various embodiments, an inadequate response to prior treatment refers to subjects with a pain symptom (e.g., any symptom listed herein) that has not improved or that has worsened despite prior treatment.

### Patient Population

As used herein, “subject” means a human subject or human patient.

An antibody as described herein is in various embodiments administered to subjects who have rheumatoid arthritis and are suffering from unacceptable pain (UP), UP in subjects with inflammation control (IC) or UP in subjects with strict RP. In various embodiments, the subject has UP and rheumatoid arthritis. In various embodiments, the subject has UP-IC and rheumatoid arthritis. In various embodiments, the subject has UP strict RP and rheumatoid arthritis. In various embodiments, the subject was previously ineffectively treated for rheumatoid arthritis by administering one or more DMARDs different from the IL-6R antibody.

A subject who is considered “ineffectively treated” by his or her physician is a subject who in various embodiments either has shown to be intolerant to the one or more DMARDs tested by the physician, and/or a subject who has shown an inadequate response to the one or more DMARDs tested by the physician, typically a subject who is still considered by the physician to present with, or to have, UP despite the previous one or more DMARDs administered.

In various embodiments, a subject with rheumatoid arthritis has:
at least 6 of 66 swollen joints and 8 of 68 tender joints, as counted by the physician in a typical quantitative swollen and tender joint count examination,
High sensitivity C-reactive protein (hs-CRP)≥8 mg/L or ESR≥28 mm/H
DAS28ESR >5.1.

In various embodiments, the subject, who was previously ineffectively treated for rheumatoid arthritis by administering at least one DMARD different from the antibody, is a subject who was previously ineffectively treated for UP by administering a DMARD. In various embodiments, the DMARD is selected from the group consisting of methotrexate, sulfasalazine, leflunomide, and hydroxychloroquine. In various embodiments, the DMARD is methotrexate. In various embodiments, the DMARD is a TNF-α antagonist. In various embodiments, the DMARD is adalimumab.

In various embodiments, the subject, who was previously ineffectively treated for UP by administering one or more DMARD different from the antibody, is a subject who had an inadequate response or intolerance to methotrexate.

In various embodiments, for those subjects previously ineffectively treated for UP by administering one or more DMARD different from the IL-6R antibody, the one or more DMARD is/are not administered anymore to the subject, and the IL-6R antibody is in various embodiments administered alone, in monotherapy to the subject.

In various embodiments, the subject is intolerant to the DMARD due to one or more physical reactions, conditions or symptoms from the treatment with the DMARD. Physical reactions, conditions or symptoms can include allergies, pain, nausea, diarrhea, azotemia, bleeding of the stomach, intestinal bleeding, canker sores, decreased blood platelets, perforation of the intestine, bacterial infection, inflammation of gums or mouth, inflammation of the stomach lining or intestinal lining, bacterial sepsis, stomach ulcer, intestinal ulcer, sun sensitive skin, dizziness, loss of appetite, low energy, and vomiting. In certain embodiments, intolerance can be determined by the subject or by a medical professional upon examination of the subject. In various embodiments, the DMARD is selected from the group consisting of methotrexate, sulfasalazine, leflunomide, and hydroxychloroquine. In certain embodiments, the DMARD is methotrexate.

In certain embodiments the disclosure provides administering to the subject one or more additional therapeutic agents in combination with the IL-6R antibody. As used herein, the expression “in combination with” means that the additional therapeutic agents are administered before, after, or concurrent with the pharmaceutical composition comprising the IL-6R antibody. In certain embodiments, the subject is administered the antibody with a DMARD and/or TNF-α antagonist.

All publications mentioned herein are incorporated herein by reference in their entirety for all purposes.

### Example

Example 1: Odds ratios for unacceptable pain (UP) and unacceptable pain despite refractory pain (RC) or inflammation control-strict (RP-strict) for three randomized controlled clinical trials (RCTs) of sarilumab.

It has been previously observed in three RCTs of sarilumab given subcutaneously at a dose of 150 mg or 200 mg every 2 weeks versus other treatments that sarilumab improved pain in RA patients.

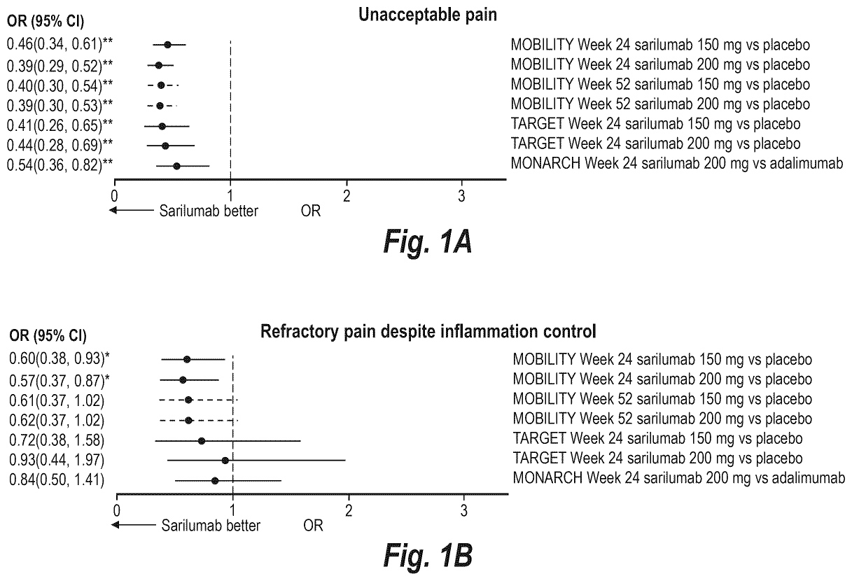

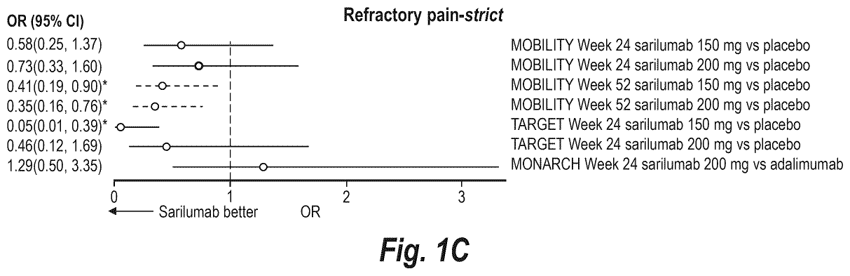

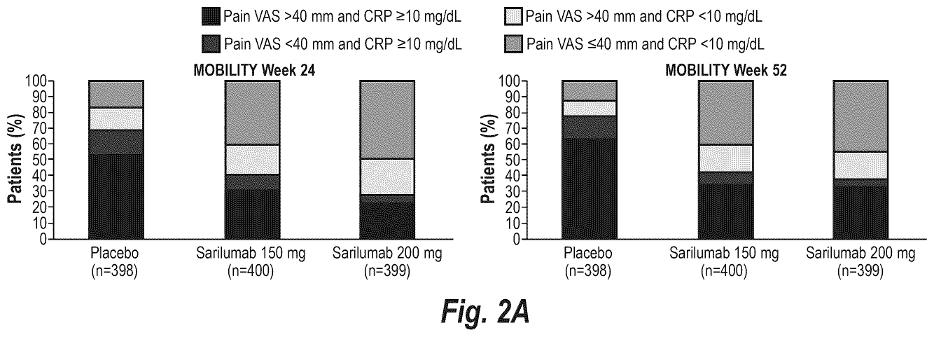

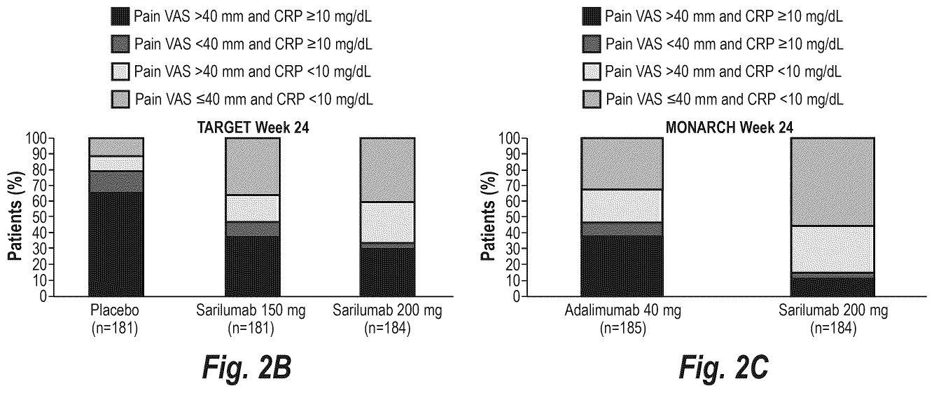

Across all three trials (with similar baseline characteristics per arm in each trial, see Table 1), sarilumab administered at 150 mg and 200 mg was associated with better inflammation control and lower rates of UP versus comparators (see FIGS. 2A-2C) and had lower odds of UP versus comparators (nominal p<0.05, See FIG. 1A-1C).

Across all three RCTs, sarilumab 150 mg and 200 mg had lower odds of unacceptable pain versus comparators with ORs (FIG. 1A):
MOBILITY, Week 24 sarilumab 150 mg: 0.46 [0.34, 0.61]; sarilumab 200 mg: 0.39 [0.29, 0.52]) and MOBILITY, Week 52 (sarilumab 150 mg: 0.40 [0.30, 0.54]; sarilumab 200 mg: 0.39 [0.30, 0.53]; all nominal p<0.001
TARGET, sarilumab 150 mg: 0.41 [0.26, 0.65]; sarilumab 200 mg: 0.44 [0.28, 0.69]); nominal p<0.05.
MONARCH, sarilumab 200 mg: 0.54 [0.36, 0.81]; nominal p<0.05

Data from the MOBILITY trial showed both sarilumab doses had lower odds (nominal p<0.05) of RP despite inflammation control when compared to placebo at Week 24 (sarilumab 150 mg: 0.60 [0.38, 0.93]; sarilumab 200 mg: 0.57 [0.37, 0.87]) and Week 52 (sarilumab 150 mg: 0.64 [0.37, 1.02]; sarilumab 200 mg: 0.62 [0.37, 1.02]), and RP-strict (MOBILITY trial) at Week 52 (sarilumab 150 mg: 0.41 [0.19, 0.90]; sarilumab 200 mg: 0.35 [0.16, 0.76]) (FIG. 1C). Data from the TARGET trial showed that sarilumab at 150 mg had lower odds (p<0.05) of RP-strict at Week 24 (0.05 [0.01; 0.39]). Higher pain level was associated with worse level of FACIT-fatigue, HAQ, SJC and TJC (all p<0.001), and UP had mostly moderate agreements with the likelihood of achieving response on minimal clinically important differences on all these outcomes (Kappa coefficient values 0.41-0.60).

There were no significant differences in the odds of refractory pain for sarilumab 200 mg versus placebo or adalimumab 40 mg in TARGET and MONARCH, respectively.

### Overall Study Design and Plan:

### Post Hoc Analysis of Three Randomized Controlled Clinical Trials (RCTs)

Data from three Phase 3 RCTs were collected to determine if sarilumab contributed to improvements in pain in subjects with RA. The MOBILITY study [NCT01061736] administered 150 mg or 200 mg sarilumab with conventional DMARDs in patients once every two weeks compare to placebo for 24 or 52 weeks; the TARGET study [NCT01709578] administered sarilumab with conventional DMARDs in patients for 24-weeks; and the MONARCH study [NCT02332590] compared patients given 200 mg sarilumab monotherapy once every two weeks to patients that received 40 mg adalimumab monotherapy once every two weeks. Post-hoc analyses were conducted on the odds rations (ORs) of pain outcomes: UP (based on patient acceptable symptom state [PASS] on a threshold of Visual Analog Scale pain>40 mm [0-100]), RP (UP C-reactive protein<10 mg/L and RP-strict (RP with swollen joint count≤1), and associations between pain and fatigue (FACIT-Fatigue) and disease activity (Health Assessment Questionnaire [HAQ], SJC, and tender joint count [TJC]). The demographics and clinical characteristics of the populations for each trial are outlined in Table 1.

TABLE 1
Demographics and clinical characteristics of trial population
MOBILITY Trial

| | Placebo + | 150 mg q2w + | 200 mg q2w + |
|---|---|---|---|
| | methotrexate | methotrexate | methotrexate |
| | (n = 398) | (n = 400) | (n = 399) |
| Age, years, mean ± | 50.9 ± 11.2 | 50.1 ± 11.9 | 50.8 ± 11.8 |
| SD | | | |
| Female, n (%) | 321 (80.7) | 319 (79.8) | 337 (84.9) |
| Race, White - | 343 (86.2) | 345 (86.3) | 343 (86.0) |
| Caucasian, n (%) | | | |
| Duration of RA, | 9.1 ± 8.1 | 9.5 ± 8.5 | 8.6 ± 7.0 |
| years, mean ± SD | | | |
| CRP, mean ± SD | 20.5 ± 23.0 | 22.5 ± 23.1 | 22.2 ± 23.8 |
| SJC (66 assessed), | 16.7 ± 9.3 | 16.6 ± 9.0 | 16.8 ± 9.7 |
| mean ± SD | | | |
| TJC, mean ± SD | 26.8 ± 13.7 | 27.2 ± 14.2 | 26.5 ± 14.5 |
| Pain-VAS, mean ± | 63.7 ± 19.9 | 65.5 ± 21.4 | 66.7 ± 21.4 |
| SD | | | |
| HAQ-DI, mean ± | 1.6 ± 0.7 | 1.6 ± 0.6 | 1.7 ± 0.6 |
| SD | | | |
| FACIT-Fatigue, | 27.2 ± 10.4 | 26.3 ± 9.8 | 25.9 ± 10.4 |
| mean ± SD | | | |

TARGET Trial

| | Placebo + | 150 mg q2w + | 200 mg q2w + |
|---|---|---|---|
| | csDMARD(s) | csDMARD(s) | csDMARD(s) |
| | (n = 181) | (n = 181) | (n = 184) |
| Age, years, mean ± | 51.9 ± 12.4 | 54.0 ± 11.7 | 52.9 ± 12.9 |
| SD | | | |
| Female, n (%) | 154 (85.1) | 142 (78.5) | 151 (82.1) |
| Race, White - | 124 (68.5) | 134 (74.0) | 130 (70.7) |
| Caucasian, n (%) | | | |
| Duration of RA, | 12.0 ± 10.0 | 11.6 ± 8.6 | 12.7 ± 96 |
| years, mean ± SD | | | |
| CRP, mean ± SD | 26.0 ± 25.2 | 23.6 ± 23.4 | 30.8 ± 28.4 |
| SJC (66 assessed), | 20.2 ± 11.3 | 19.6 ± 11.2 | 20.0 ± 11.9 |
| mean ± SD | | | |
| TJC, mean ± SD | 29.4 ± 14.5 | 27.7 ± 15.6 | 29.6 ± 15.5 |
| Pain-VAS, mean ± | 71.6 ± 18.2 | 71.0 ± 19.4 | 74.9 ± 18.4 |
| SD | | | |
| HAQ-DI, mean ± | 1.8 ± 0.6 | 1.7 ± 0.6 | 1.8 ± 0.6 |
| SD | | | |
| FACIT-Fatigue, | 23.7 ± 10.8 | 23.5 ± 10.6 | 23.1 ± 10.8 |
| mean ± SD | | | |

MONARCH Trial

| | Adalimumab SC 40 mg q2w/qw | Sarilumab SC 200 mg q2w |
|---|---|---|
| | (n = 185) | (n = 184) |
| Age, years, mean ± | 53.6 ± 11.9 | 50 ± 12.6 |
| SD | | |
| Female, n (%) | 150 (81.1) | 157 (85.3) |
| Race, White - | 164 (88.6) | 171 (92.9) |
| Caucasian, n (%) | | |
| Duration of RA, | 6.6 ± 7.8 | 8.1 ± 8.1 |
| years, mean ± SD | | |
| CRP, mean ± SD | 24.1 ± 31.0 | 17.4 ± 21.3 |
| SJC (66 assessed), | 17.5 ± 10.3 | 18.6 ± 10.7 |
| mean ± SD | | |
| TJC, mean ± SD | 26.7 ± 13.6 | 28.0 ± 13.2 |
| Pain-VAS, mean ± | 71.4 ± 18.96 | 71.6 ± 18.65 |
| SD | | |
| HAQ-DI, mean ± | 1.6 ± 0.64 | 1.6 ± 0.55 |
| SD | | |
| FACIT-Fatigue, | 24.0 ± 10.31 | 23.6 ± 9.01 |
| mean ± SD | | |

Abbreviations: qw = once every week; q2w = once every other week; SC = subcutaneous; HAQ-DI = Health Assessment Questionnaire Disability Index; SD = Standard Deviation

1. A method for treating unacceptable pain (UP) in a subject in need thereof who has rheumatoid arthritis, comprising administering, to a subject who is determined to have rheumatoid arthritis and UP, an antibody that specifically binds IL-6 receptor, and a disease modifying antirheumatic drug (DMARD) that is different than the antibody,
wherein the UP has a level of pain indicated by a visual analogy scale (VAS) of greater than 40 mm; and
wherein the antibody comprises a heavy chain variable region comprising complementarity determining regions HCDR1, HCDR2, and HCDR3 of a heavy chain variable region comprising the amino acid sequence of SEQ ID NO: 1 and complementary determining regions LCDR1, LCDR2, and LCDR3 of a light chain variable region comprising the amino acid sequence of SEQ ID NO: 2.

2. The method of claim 1, wherein the antibody comprises a heavy chain variable region comprising the amino acid sequence of SEQ ID NO: 1 and a light chain variable region comprising the amino acid sequence of SEQ ID NO: 2.

3. (canceled)

4. (canceled)

5. The method of claim 1, wherein the subject experiences a reduction in VAS to less than 40 mm after 24 or 54 weeks of treatment.

6. (canceled)

7. The method of claim 1, wherein the subject, prior to the administering the antibody, has a Disease Activity Score (DAS) of from 3.2 to 5.1, or has a DAS of greater than 5.1.

8. (canceled)

9. (canceled)

10. The method of claim 1, wherein the subject is administered subcutaneously a dose of about 150 mg or about 200 mg of the antibody every two weeks.

11-17. (canceled)

18. The method of claim 1, wherein the DMARD different than the antibody comprises methotrexate or a TNF antagonist.

19. (canceled)

20. (canceled)

21. The method of claim 1, wherein the subject (i) was previously ineffectively treated for rheumatoid arthritis by administering at least one DMARD different from the antibody, (ii) is intolerant of at least one DMARD different from the antibody, or (iii) is considered an inappropriate candidate for continued treatment of rheumatoid arthritis by administering at least one DMARD different from the antibody.

22. The method according to claim 21, wherein the at least one DMARD different from the antibody comprises methotrexate or a TNF-α antagonist.

23-29. (canceled)

30. A method for treating unacceptable pain (UP) in a subject in need thereof who has rheumatoid arthritis, comprising
administering to a subject who is determined to have rheumatoid arthritis and UP despite inflammation control an antibody that specifically binds IL-6 receptor and a disease modifying antirheumatic drug (DMARD),
wherein the UP has a level of pain indicated by a visual analogy scale (VAS) of greater than 40 mm;
wherein the antibody comprises complementarity determining regions HCDR1, HCDR2, and HCDR3 of a heavy chain variable region comprising the amino acid sequence of SEQ ID NO: 1 and complementary determining regions LCDR1, LCDR2, and LCDR3 of a light chain variable region comprising the amino acid sequence of SEQ ID NO: 2.

31. The method of claim 30, wherein the antibody comprises a heavy chain variable region comprising the amino acid sequence of SEQ ID NO: 1 and a light chain variable region comprising the amino acid sequence of SEQ ID NO: 2.

32. The method of claim 30, wherein the subject is determined to have refractory pain (RP) or strict RP.

33. (canceled)

34. The method of claim 30, wherein the subject experiences a reduction in VAS to less than 40 mm after 24 or 54 weeks of treatment.

35. (canceled)

36. The method of claim 30, wherein the subject, prior to the administering the antibody and the DMARD, has a Disease Activity Score (DAS) of from 3.2 to 5.1 or has a DAS of greater than 5.1.

37. (canceled)

38. (canceled)

39. The method of claim 1, wherein the subject is administered subcutaneously a dose of about 150 mg or about 200 mg of the antibody every two weeks.

40. (canceled)

41. The method of claim 1, wherein inflammation control is inflammation in the subject that had been reduced by a DMARD selected from one or more of methotrexate, etanercept, infliximab, adalimumab, golimumab and certolizumab pegol.

42-87. (canceled)

88. A method for treating unacceptable pain (UP) in a subject in need thereof who has rheumatoid arthritis, comprising administering to a subject who is determined to have rheumatoid arthritis, UP, and a serum level of C-reactive protein (CRP) of less than 10 mg/L, an antibody that specifically binds to an IL-6 receptor and a disease modifying antirheumatic drug (DMARD),
wherein the antibody comprises complementarity determining regions HCDR1, HCDR2, and HCDR3 of a heavy chain variable region comprising the amino acid sequence of SEQ ID NO: 1 and complementary determining regions LCDR1, LCDR2, and LCDR3 of a light chain variable region comprising the amino acid sequence of SEQ ID NO: 2, wherein UP has a level of pain indicated by a visual analog scale (VAS) score of greater than 40 mm.

89. The method of claim 88, wherein the antibody comprises a heavy chain variable region comprising the amino acid sequence of SEQ ID NO: 1 and a light chain variable region comprising the amino acid sequence of SEQ ID NO: 2.

90. The method of claim 88, wherein the subject is further determined to have a swollen joint count (SJC) of equal to or less than 1.

91. The method of claim 88, wherein the subject is administered subcutaneously a dose of about 150 mg or about 200 mg of the antibody every two weeks.

92. The method of claim 1, wherein the (a) HCDR1 comprises the amino acid sequence of SEQ ID NO: 3; (b) HCDR2 comprises the amino acid sequence of SEQ ID NO: 4; (c) HCDR3 comprises the amino acid sequence of SEQ ID NO: 5; (d) LCDR1 comprises the amino acid sequence of SEQ ID NO: 6; (e) LCDR2 comprises the amino acid sequence of SEQ ID NO: 7; and (f) LCDR3 comprises the amino acid sequence of SEQ ID NO: 8.

93. The method of claim 30, wherein the (a) HCDR1 comprises the amino acid sequence of SEQ ID NO: 3; (b) HCDR2 comprises the amino acid sequence of SEQ ID NO: 4; (c) HCDR3 comprises the amino acid sequence of SEQ ID NO: 5; (d) LCDR1 comprises the amino acid sequence of SEQ ID NO: 6; (e) LCDR2 comprises the amino acid sequence of SEQ ID NO: 7; and (f) LCDR3 comprises the amino acid sequence of SEQ ID NO: 8.

94. The method of claim 88, wherein the (a) HCDR1 comprises the amino acid sequence of SEQ ID NO: 3; (b) HCDR2 comprises the amino acid sequence of SEQ ID NO: 4; (c) HCDR3 comprises the amino acid sequence of SEQ ID NO: 5; (d) LCDR1 comprises the amino acid sequence of SEQ ID NO: 6; (e) LCDR2 comprises the amino acid sequence of SEQ ID NO: 7; and (f) LCDR3 comprises the amino acid sequence of SEQ ID NO: 8


SEQUENCE LISTING (11 sequences)

SEQ ID NO: 1 - protein, 116 aa, synthetic construct
  REGION 1..116: note = Heavy chain variable region
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 2 - protein, 107 aa, synthetic construct
  REGION 1..107: note = Light chain variable region
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 3 - protein, 8 aa, synthetic construct
  REGION 1..8: note = HCDR1
RFTFDDYA

SEQ ID NO: 4 - protein, 8 aa, synthetic construct
  REGION 1..8: note = HCDR2
ISWNSGRI

SEQ ID NO: 5 - protein, 9 aa, synthetic construct
  REGION 1..9: note = HCDR3
AKGRDSFDI

SEQ ID NO: 6 - protein, 6 aa, synthetic construct
  REGION 1..6: note = LCDR1
QGISSW

SEQ ID NO: 7 - skipped

SEQ ID NO: 8 - protein, 9 aa, synthetic construct
  REGION 1..9: note = LCDR3
QQANSFPYT

SEQ ID NO: 9 - protein, 446 aa, synthetic construct
  REGION 1..446: note = Heavy chain
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = Light chain
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 358 aa, synthetic construct
  REGION 1..358: note = Extracellular domain of hIL-6R
MVAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQD